(19) United States
(12) Patent Application Publication
(10) Pub. No.: US 2025/0378933 A1
(43) Pub. Date: Dec. 11, 2025

(54) FOOD SAFETY AND NUTRITIONAL LABELING ASSISTANCE METHODS AND SYSTEMS

(71) Applicant: ESHA Research, LLC, dba Trustwell, Beaverton, OR (US)

(72) Inventors: Douglas James Bell, Happy Valley, OR (US); Todd Dolinsky, Chapel Hill, NC (US)

(21) Appl. No.: 19/228,629

(22) Filed: Jun. 4, 2025

Related U.S. Application Data
(60) Provisional application No. 63/656,460, filed on Jun. 5, 2024.

(51) Int. Cl. G16H 20/60; G06F 16/334

(52) U.S. Cl. CPC G16H 20/60; G06F 16/334

(57) ABSTRACT

Techniques for providing food safety or nutritional labeling guidance to a user are provided. In one embodiment, a method includes electronically receiving a natural language prompt regarding food safety or nutritional labeling from a user via a network and sending a response request based on the received natural language prompt regarding food safety or nutritional labeling to a trained large language model that has been trained with a data training set including food safety or nutritional labeling reference data. The method also includes receiving a natural language response from the trained large language model in reply to the response request and providing the natural language response to the user via the network. Additional methods, systems, and devices are also disclosed.

### BACKGROUND

This section is intended to introduce the reader to various aspects of art that may be related to various aspects of the presently described embodiments. This discussion is believed to be helpful in providing the reader with background information to facilitate a better understanding of the various aspects of the present embodiments. Accordingly, it should be understood that these statements are to be read in this light, and not as admissions of prior art.

Food is essential to civilization, and governments have been regulating food for centuries. Governments enact and enforce standards regarding food for various purposes, such as to maintain quality, prevent contamination, avoid food-borne illnesses, and provide information to consumers. Different jurisdictions have a patchwork of different laws, regulations, and practices regarding food safety and labeling. Among other things, regulations may place limits on contaminants and residues in food and mandate disclosure of ingredients and nutritional information. To ensure compliance, companies in the food industry can search for relevant laws, regulations, or other authority in a jurisdiction. Past efforts to facilitate regulatory compliance for the food industry in the United States include maintaining a United States Code of Federal Regulation (CFR) Title 21 search system that is searchable via a Boolean search. But food safety and nutritional labeling laws, regulations, and practices change over time, frustrating compliance.

### SUMMARY

Certain aspects of some embodiments disclosed herein are set forth below. It should be understood that these aspects are presented merely to provide the reader with a brief summary of certain forms the invention might take and that these aspects are not intended to limit the scope of the invention. Indeed, the invention may encompass a variety of aspects that may not be set forth below.

Certain embodiments of the present disclosure generally relate to food safety or nutritional labeling. More specifically, at least some embodiments include a virtual assistant for helping users navigate the complexities of regulations in the food, food safety, and supplement manufacturing industries. The virtual assistant can be engineered to understand and respond to queries in natural language, simplifying the search for relevant regulations or other authority by a user. By way of example, the virtual assistant can use a large language model (LLM) that has been trained to reply to questions using natural language to give conversational answers in a manner similar to a human expert.

Various refinements of the features noted above may exist in relation to various aspects of the present embodiments. Further features may also be incorporated in these various aspects as well. These refinements and additional features may exist individually or in any combination. For instance, various features discussed below in relation to one or more of the illustrated embodiments may be incorporated into any of the above-described aspects of the present disclosure alone or in any combination. Again, the brief summary presented above is intended only to familiarize the reader with certain aspects and contexts of some embodiments without limitation to the claimed subject matter.

### BRIEF DESCRIPTION OF THE DRAWINGS

These and other features, aspects, and advantages of certain embodiments will become better understood when the following detailed description is read with reference to the accompanying drawings in which like characters represent like parts throughout the drawings, wherein:

FIG. 1 generally depicts a networked computing environment for deploying applications in accordance with one embodiment of the present disclosure;

FIG. 2 depicts an architecture of an application for providing food safety or nutritional labeling guidance to a user in accordance with one embodiment;

FIG. 3 is a diagram depicting components and operation of a computer system using a trained large language model for providing food safety or nutritional labeling guidance to a user in accordance with one embodiment;

FIG. 4 is a flowchart representing a method for shaping a natural language prompt received from a user in accordance with one embodiment;

FIG. 5 is a flowchart representing a method for shaping a natural language response received from the trained large language model in accordance with one embodiment;

FIG. 6 is a diagram representing retrieval of user history by the computer system of FIG. 3 in accordance with one embodiment;

FIG. 7 is a diagram depicting training of the large language model in accordance with one embodiment; and

FIG. 8 is a block diagram of components of a programmed computer system for providing food safety or nutritional labeling guidance to a user in accordance with one embodiment.

### DETAILED DESCRIPTION OF SPECIFIC EMBODIMENTS

Specific embodiments of the present disclosure are described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time-consuming, but would nevertheless be a routine undertaking of design, fabrication, and manufacture for those of ordinary skill having the benefit of this disclosure.

When introducing elements of various embodiments, the articles “a,” “an,” “the,” and “said” are intended to mean that there are one or more of the elements. The terms “comprising,” “including,” and “having” are intended to be inclusive and mean that there may be additional elements other than the listed elements.

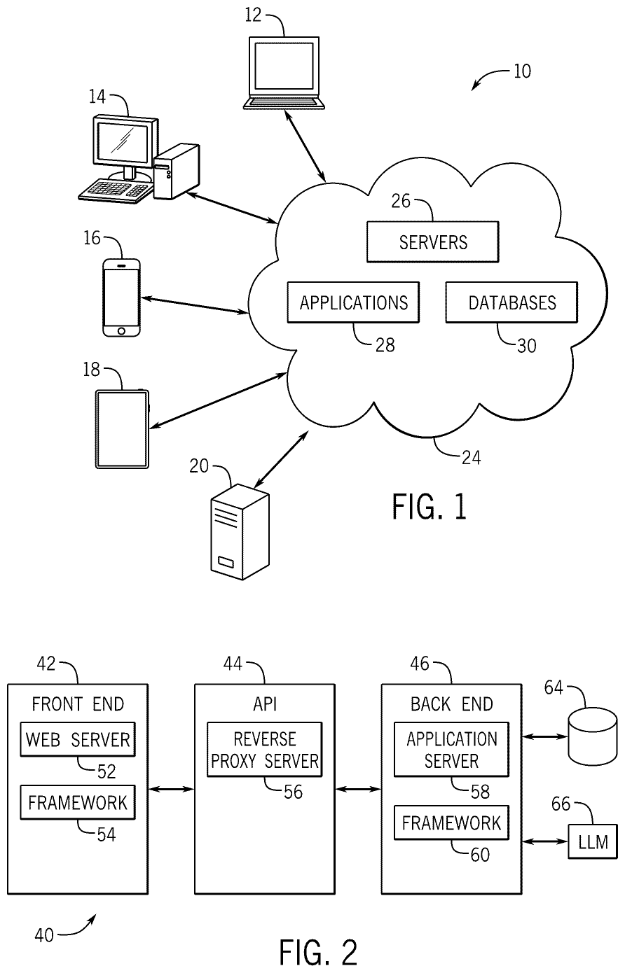

Turning now to the present figures, FIG. 1 shows an example of an electronic system 10 in the form of a networked computing environment. In this depicted embodiment, the system 10 includes various endpoint devices, such as a laptop computer 12, a desktop computer 14, a smartphone 16, a tablet 18, and a server 20. These endpoints communicate with a network 24 that may include one or more servers 26, software applications 28, and databases 30 to provide services to a user. Various devices of the system 10 may be local or remote and can communicate with other devices via any suitable communication protocols. In some embodiments, the network 24 is a cloud computing environment accessed by an endpoint device via the Internet. In other instances, however, the network 24 could be a local network provided on site with one or more of the endpoint devices.

Various applications 28 can be deployed via the network 24. In some embodiments, an application 28 of the electronic system 10 is an executable software program that includes a virtual assistant for providing regulatory or other guidance for the food, food safety, and supplement manufacturing industries. The virtual assistant can be engineered to understand and respond to queries in natural language, simplifying the search for relevant regulations or other authority by a user. In some embodiments, the virtual assistant may be used in performing a computer-implemented method of providing food safety or nutritional labeling guidance to a user. In one embodiment, for example, such a method may include electronically receiving a natural language prompt regarding food safety or nutritional labeling from the user via a network, sending a response request based on the received natural language prompt regarding food safety or nutritional labeling to a trained large language model, receiving a natural language response from the trained large language model in reply to the response request, and providing the natural language response to the user via the network.

In some instances, such an application 28 for providing food safety or nutritional labeling guidance to a user is a web application. The web application can be deployed via a cloud computing platform, such as Amazon Web Services (AWS) or Microsoft Azure, or in any other suitable manner. By way of example, an architecture of such a web application is depicted in FIG. 2 in accordance with one embodiment. It will be appreciated, however, that the architecture and implementation of such a web application may differ in other embodiments.

In FIG. 2, the web application 40 is depicted as a three-tier web application with front end 42, application programming interface (API) 44, and back end 46 services. The front end 42 includes a web server 52 and framework 54, the API 44 includes a reverse proxy server 56, and the back end 46 includes an application server 58 and framework 60. In at least one embodiment, the web application 40 is built using Java for the back-end logic with Spring Boot to streamline application development, Angular and TypeScript for the front-end user interface, and NGINX as a high-performance server and reverse proxy. For instance, NGINX can be used for the web server 52 to efficiently handle incoming traffic to the web front end 42 and Angular can be used for the framework 54 to build the client side (user interface) of the web application 40 as a dynamic single-page application (SPA). NGINX can also serve as the reverse proxy server 56 for the web application's API endpoints, ensuring efficient management of the traffic between clients and the application server 58. Java can be the primary programming language for the back-end application logic, Apache Tomcat can be used as the application server 58 to serve the API 44 (which may also be Java-based), and Spring Boot can be used as the framework 60.

Data used or generated by the web application 40 can be stored in a database 64. Although the database 64 could also or instead be stored in one or more local storage devices, in at least some instances the database 64 is a cloud database. For example, application data can be stored in MongoDB Atlas and the web application 40 can use a non-relational database for efficient data storage of chat history and other application data.

The web application 40 leverages a large language model 66 for providing guidance to user inquiries. The large language model 66 can be trained with a data training set including food safety and nutritional labeling reference data so that the large language model 66 can reply using natural language to give conversational answers to questions from users regarding food safety and nutritional labeling. Non-limiting examples of such reference data include various authority related to food safety or nutritional labeling, such as any or all of pertinent regulations, codes, statutes, and legislation, any of which could be currently in effect, due to come into effect, or proposed for future effect. The authority may also include data on procedures, interpretations, directives, guidance, notices, or programs of one or more agencies or other governmental organizations. For the United States, these may include the Food and Drug Administration (FDA), the Food Safety and Inspection Service (FSIS) of the Department of Agriculture, or the Center for Disease Control (CDC). Further, the authority may include mandatory or voluntary recall notices. While the large language model 66 could be trained on authority from a single legal jurisdiction (e.g., the United States or the European Union), in some embodiments the large language model 66 is trained on nutritional labeling and food safety regulations from multiple jurisdictions.

By way of example, in one embodiment the large language model 66 can be trained using Title 21 of the United States Code of Federal Regulations (e.g., Parts 101 and 111) and laws and regulations administered by the FSIS (e.g., Federal Meat Inspection Act, Poultry Products Inspection Act, Egg Products Inspection Act, and Humane Methods of Slaughter Act) as training data. In some instances, the large language model 66 is also trained using some or all of the training data indicated in the table below:

| Jurisdiction | Laws/Regulations | Additional Authority |
|---|---|---|
| Australia | Food Standards | Food Standards (Proposal P293 - |
| | Code | Pregnancy warning labels on |
| | | alcohol products) |
| | | Food Standards (Proposal P1025 - |
| | | Country of Origin Labelling) |
| Canada | Food and Drugs Act | Food and Drug Regulations |
| | | (Division 24 - Nutrition Labelling) |
| European | Regulation (EU) No. | Commission Delegated Regulation |
| Union | 1169/2011 - Food | (EU) No. 78/2014 (Supplementing |
| | Information to | Regulation (EU) No. 1169/2011) |
| | Consumers | Regulation (EU) No. 1924/2006 |
| | | (Nutrition and Health Claims) |
| Mexico | Ley General de Salud | Norma Oficial Mexicana NOM- |
| | (General Health | 051-SCFI/SSA1-2010 (General |
| | Law) | Labelling of Prepackaged Goods - |
| | | Nutrition Labelling) |
| New Zealand | Food Act 2014 | Food (Standard, Transitional |
| | | and General Food) Regulations |
| | | 2015 |
| United States | Food Safety | Food Labeling Modernization Act |
| | Modernization | of 2021 (proposed) |
| | Act (FSMA) | |
| United States | Nutrition Labeling | Dietary Supplement Health and |
| | and Education Act | Education Act (DSHEA) |
| | (NLEA) | Food Allergen Labeling and |
| | | Consumer Protection Act |
| | | (FALCPA) |

Any suitable parameters or hyperparameters may be used during training of the large language model 66. In one example, the large language model 66 is an OpenAI model trained using Batch Size set to 32, Learning Rate Multiplier set to 1, and Number of Epochs set to 1. But any other suitable models, parameters, and hyperparameters could be used in full accordance with the present techniques. Training may be supervised or unsupervised and carried out in any suitable manner. And while a single large language model 66 can be trained using authority from multiple jurisdictions, multiple large language models 66 could be used in other instances (e.g., training each of multiple large language models using reference authority from a different single jurisdiction).

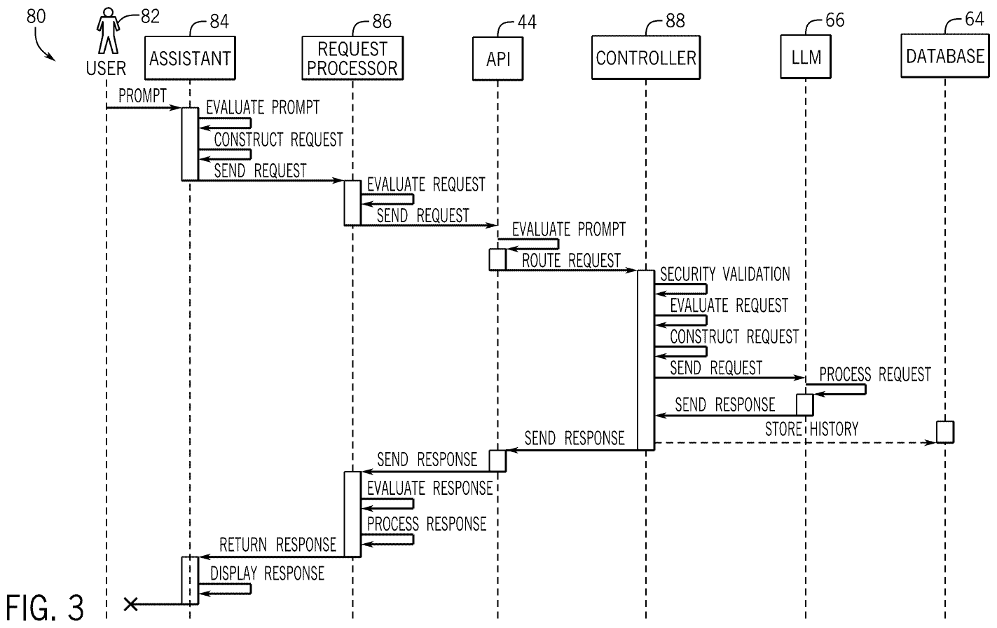

An example of components and operation of a computer system 80 using the trained large language model for providing food safety or nutritional labeling guidance to a user 82 is provided in FIG. 3 in accordance with one embodiment. As presently depicted, the system 80 includes a virtual assistant 84 (such as described above), a request processor 86, the API 44, and a controller 88. These components can be software (e.g., the web application 40) executed by one or more computers, which in at least some embodiments are part of a cloud computing environment. The user 82 can electronically transmit a natural language prompt regarding food safety or nutritional labeling from a processor-based client device (e.g., one of the endpoint devices of FIG. 1 connected to the network 24) to the assistant 84.

The assistant 84 is configured to initiate a response request for the large language model 66 based on the natural language prompt. As shown in FIG. 3, initiating the response request can include the assistant 84 evaluating the received natural language prompt, constructing the response request, and sending the constructed response request to the trained large language model 66 (via request processor 86, API 44, and controller 88).

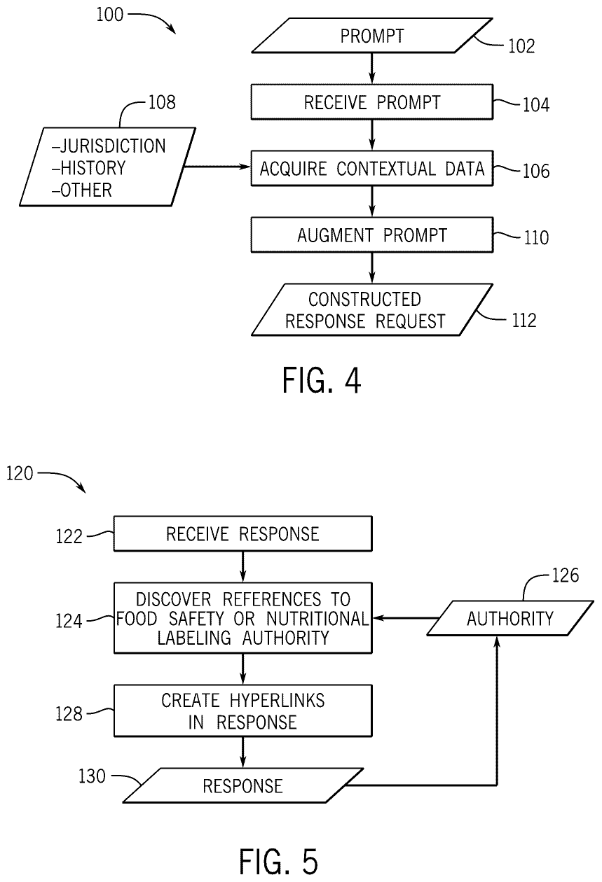

In at least some instances, constructing the response request includes shaping the natural language prompt received from the user 82. One example of shaping the natural language prompt is generally provided by flowchart 100 in FIG. 4 in accordance with one embodiment. In this example, the natural language prompt 102 regarding food safety or nutritional labeling is received (block 104) and contextual data 108 related to the prompt 102 is acquired (block 106). The contextual data 108 may include a relevant jurisdiction, history, or other data. In some instances, acquiring the contextual data 108 includes identifying the jurisdiction applicable to the natural language prompt, which may include receiving a user-selected indication of the jurisdiction (e.g., the user may select the applicable jurisdiction via a user interface of the web application 40). But the jurisdiction applicable to the natural language prompt could be acquired in any other suitable fashion. For example, an internet protocol (IP) address of the user can be used to determine a user location (which may be assumed to be the jurisdiction of interest absent an indication to the contrary), a default jurisdiction may be used (e.g., from a user record), or the natural language prompt may itself specify a jurisdiction to which the prompt is directed (which may or may not be the jurisdiction in which the user is located). Historical contextual data may include previous prompts from a user in the current session, previous replies from the large language model 66 provided to the user in the current session, user conversation history preceding a current session (e.g., a chat log), or other user data accessible to the web application 40.

The prompt may then be augmented (block 110) based on the acquired contextual data 108 to produce a constructed response request 112. By way of example, based on an identified jurisdiction applicable to the natural language prompt 102, the assistant 84 can augment the prompt by providing a system level message to the large language model 66 as to which authority to use in responding to the prompt 102. For instance, following an identification of the United States as the applicable jurisdiction, the constructed response request 112 may include the prompt 102, as well as a system-added instruction for the large language model 66, such as: “You will only use the FDA Food Labeling and dietary supplements Regulations (21 CFR Part 101 and Part 111), the Food Safety Modernization Act (FSMA), and the USDA Food Safety and Inspection Service (FSIS) you have been trained on to reply to the following prompts.” Augmenting the prompt may also or instead include adding historical or other contextual data to the constructed response request 112 or modifying the prompt based on such data.

Returning now to FIG. 3, the request processor 86 evaluates the constructed response request received from the assistant 84 and sends the request to the API 44, which may also evaluate the request and then route the request to the controller 88. It will be appreciated that the constructed response request may be sent and routed in any suitable manner. In some cases, this may be based on various paths or parameters; for instance, specific routing of the constructed response request may vary depending on whether a prompt is part of a new conversation or is a follow-on prompt that adds to a previous prompt or conversation, on the jurisdiction applicable to the prompt, or on the location of the user, to name just a few examples. Also, feedback requests may be routed differently than prompts seeking food safety or nutritional labeling guidance. The controller 88 can perform a security validation of the request received from the API 44, evaluate the request, and then construct and send a request to the large language model 66, which in turn processes the request and sends a natural language response in reply to the response request from the assistant 84. The controller 88 receives the natural language response from the large language model 66, may store a copy of the response in the database 64, and passes the response to the API 44, which may similarly pass the response on to the request processor 86.

The request processor 86 can evaluate and process the natural language response received from the API 44 before passing the response to the assistant 84 for output to the user 82. In some embodiments, returning responses are evaluated to determine if the large language model 66 has responded with authority (e.g., codes, statutes, regulations, or proposals) from any of the regulatory bodies or other sources of authority of which it is aware. This evaluation and processing may include augmenting or otherwise shaping the received response before passing the response to the assistant 84 or before outputting the response to the user 82. An example of shaping the natural language response is generally provided by flowchart 120 in FIG. 5 in accordance with one embodiment. In this example, the natural language response is received (block 122) by the request processor 86 and electronically evaluated to discover one or more references (block 124) to food safety or nutritional labeling authority 126 within the response. Such authority may include specific regulations, codes, statutes, legislation, procedures, interpretations, directives, guidance, notices, programs, or recall notices, to name some examples, and may be authority included in the data training set used to train the large language model 66. This discovery may include token-based text searching that compares a group of regular expressions indicative of authority (e.g., “21 C.F.R.”, “21 U.S.C.”, “§ ”, “Title”, “Section”, and “Regulation No.”) to returned responses to identify references to authority.

The response may be augmented by creating hyperlinks (block 128) in the natural language response to link each of the one or more discovered references to a location for a specific portion of the food safety or nutritional labeling authority. This may include querying a database containing the authority to find a section or portion of the authority cited in a response and then linking the citation to a location (e.g., address) having that section or portion. The discovered references may also or instead be highlighted or otherwise modified to help a user 82 identify authority cited within the response. The shaped natural language response 130 may be provided electronically to the user 82 via the assistant 84. The user 82 may use the hyperlinks to cross-check responses or make direct references to the actual regulations or other authority 126 cited in the response, allowing the user 82 to vet and confirm the guidance supplied by the large language model 66.

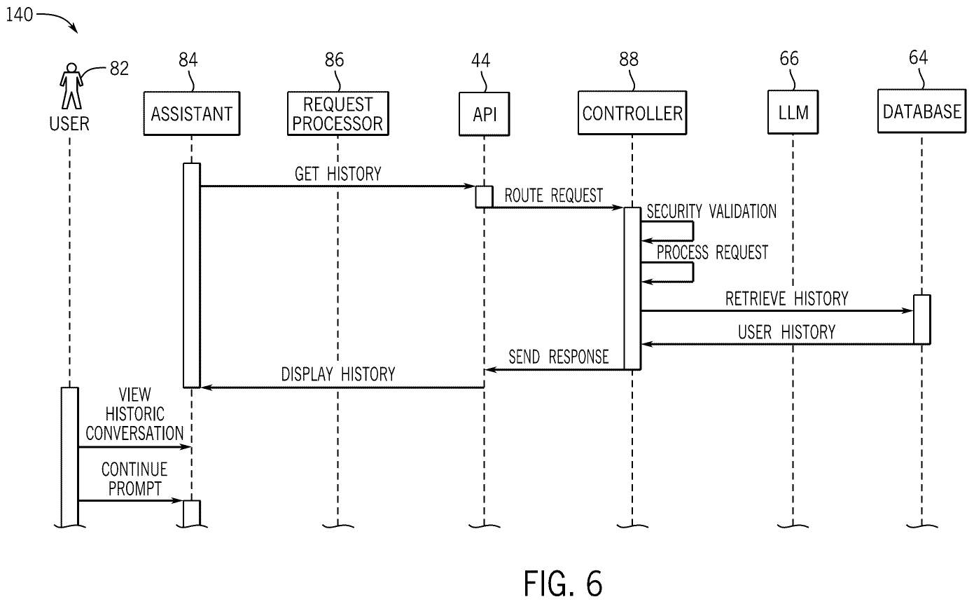

In some embodiments, a user's previous prompts or conversations (containing prompts and replies) may be stored and retrieved, such as to allow a user to refer back to or continue past conversations. The system may be context-aware when natural language responses are requested or returned from the large language model 66 and, as noted above, such previous prompts or conversations may be used by the system for context. One example of retrieving user history (e.g., past conversations) via the system 80 is shown in diagram 140 of FIG. 6. In this example, the virtual assistant 84 can request user history via the API 44, which routes the request to the controller 88. After security validation of the request, the controller 88 can process the request, retrieve the requested history from the database 64, and send a response with the requested history to the assistant 84 via the API 44. The retrieved history may be displayed to the user 82 (e.g., in a browser of a client device) by the assistant 84. In some instances, the user 82 viewing the history may edit past prompts, copy or paste from past conversations into new prompts, or request that the system 80 generate a new response. The user 82 may continue a prompt, which may be routed to the large language model 66 for reply, such as described above with respect to FIG. 3. The assistant 84 may also be configured to allow the user 82 to provide feedback on responses received from the large language model 66. This feedback could be provided in any suitable form, such as a binary indication of whether a response was helpful or user comments on a given response.

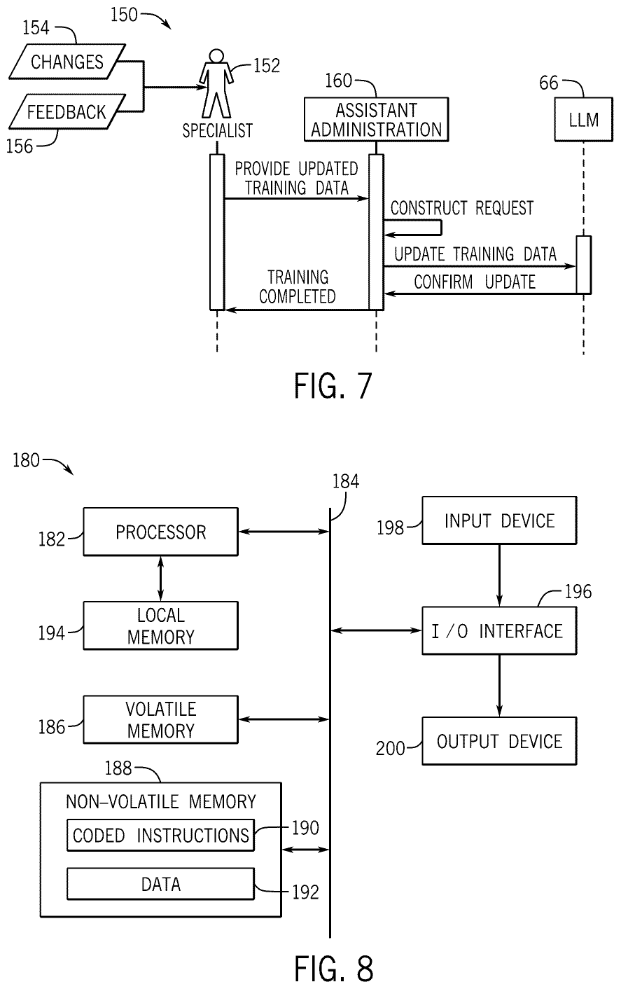

As noted above, training of the large language model 66 can be performed in any suitable manner. In some instances, updated training data may be used for further training of the large language model 66. One such example is generally depicted in diagram 150 of FIG. 7. A specialist 152, such as a human user with expertise in food safety or nutritional labeling regulations, can receive changes 154, which may include new or updated authority (e.g., new or amended laws or regulations or new food safety recalls). The specialist 152 may also review feedback 156, such as feedback from users 82 about responses received from the large language model 66. In some cases, the specialist 152 may evaluate the feedback 156, the response to which the feedback relates, and other context (e.g., the prompt to which the response is given or previous conversation data) to assess the given response. This may include determining whether the given response is accurate and complete. In some instances, the specialist 152 or another expert may provide an improved response to a prompt and that improved response can be used to further train the model. The specialist 152 can provide updated training data, such as additional training data based on the changes 154 or feedback 156, to an administration function 160 for the virtual assistant 84, which may construct a request and train the large language model 66 with the updated training data. The large language model 66 can confirm the updated training and the specialist 152 can be notified that the training is completed.

Finally, those skilled in the art will appreciate that the present techniques may be implemented via computers or other processor-based devices programmed to facilitate performance of the above-described processes. Examples of such devices include the servers 26 and client endpoint devices described above. One example of such a processor-based computer system is generally depicted in FIG. 6 in accordance with one embodiment. In this example, a computer system 180 includes a processor 182 connected via a bus 184 to volatile memory 186 (e.g., random-access memory) and non-volatile memory 188 (e.g., a hard drive, flash memory, or read-only memory (ROM)). Coded application instructions 190 and data 192 are stored in the non-volatile memory 188. The instructions 190 and the data 192 may also be loaded into the volatile memory 186 (or in a local memory 194 of the processor) as desired, such as to reduce latency and increase operating efficiency of the computer 180. The coded application instructions 190 can be provided as software that may be executed by the processor 182 to enable various functionalities described herein. Non-limiting examples of these functionalities include electronically receiving a natural language prompt regarding food safety or nutritional labeling from the user via a network, sending a response request based on the received natural language prompt regarding food safety or nutritional labeling to a trained large language model, receiving a natural language response from the trained large language model in reply to the response request, and providing the natural language response to the user via the network. Further examples of these functionalities include shaping prompts and responses, such as described above.

In at least some embodiments, the application instructions 190 are encoded in a non-transitory computer readable storage medium, such as the volatile memory 186, the non-volatile memory 188, the local memory 194, or a portable storage device (e.g., a flash drive or a compact disc). In some instances, the computer system 180 is part of a cloud computing environment, the processor 182 includes one or multiple processors of one or more servers 26, the coded application instructions 190 (e.g., of the web application 40) are stored in one or more memory devices of the cloud computing environment (e.g., a drive of one or more servers 26), and the data 192 may be stored in a database 30 or 64 (which may also be stored in one or more cloud storage devices). As used herein, the term “memory having computer-readable instructions” includes a single memory having the computer-readable instructions, multiple memories each having the computer-readable instructions, and multiple memories each having a portion of the computer-readable instructions (i.e., a set of instructions may be distributed across multiple memories).

An interface 196 of the computer system 180 enables communication between the processor 182 and various input devices 198 and output devices 200. The interface 196 can include any suitable device that enables this communication, such as a network interface card, wireless radio, modem, connector, or serial port. In some embodiments, the input devices 198 include a keyboard and a mouse to facilitate user interaction, while the output devices 200 include displays, printers, and storage devices that allow output of data received or generated by the computer system 180. Input devices 198 and output devices 200 may be provided as part of the computer system 180 or may be separately provided. It will be appreciated that computer system 180 may be a distributed system, in which some of its various components are located remote from one another (e.g., servers 26 of a cloud computing environment remote from client endpoint devices), in at least some instances.

While the aspects of the present disclosure may be susceptible to various modifications and alternative forms, specific embodiments have been shown by way of example in the drawings and have been described in detail herein. But it should be understood that the invention is not intended to be limited to the particular forms disclosed. Rather, the invention is to cover all modifications, equivalents, and alternatives falling within the spirit and scope of the invention as defined by the following appended claims.

1. A computer-implemented method of providing food safety or nutritional labeling guidance to a user, the method comprising:
electronically receiving a natural language prompt regarding food safety or nutritional labeling from the user via a network;
sending a response request based on the received natural language prompt regarding food safety or nutritional labeling to a trained large language model, wherein the trained large language model has been trained with a data training set including food safety or nutritional labeling reference data;
receiving a natural language response from the trained large language model in reply to the response request; and
providing the natural language response to the user via the network.

2. The method of claim 1, wherein sending the response request based on the received natural language prompt regarding food safety or nutritional labeling to the trained large language model includes evaluating the received natural language prompt regarding food safety or nutritional labeling, constructing the response request, and sending the constructed response request to the trained large language model.

3. The method of claim 2, wherein constructing the response request includes shaping the received natural language prompt regarding food safety or nutritional labeling.

4. The method of claim 3, wherein shaping the received natural language prompt regarding food safety or nutritional labeling includes adding contextual data with the natural language prompt in the constructed response request.

5. The method of claim 4, comprising identifying a jurisdiction applicable to the natural language prompt.

6. The method of claim 5, wherein identifying the jurisdiction applicable to the natural language prompt includes receiving a user-selected indication of the jurisdiction.

7. The method of claim 2, wherein constructing the response request includes adding historical context for the user with the natural language prompt.

8. The method of claim 1, comprising shaping the natural language response received from the trained large language model, wherein providing the natural language response to the user via the network includes providing the shaped natural language response to the user via the network.

9. The method of claim 1, wherein the food safety or nutritional labeling reference data with which the large language model has been trained includes regulatory data.

10. The method of claim 1, wherein electronically receiving the natural language prompt regarding food safety or nutritional labeling from the user via the network includes electronically receiving the natural language prompt regarding food safety or nutritional labeling from the user via a web application.

11. A computer-implemented method of acquiring food safety or nutritional labeling guidance by a user, the method comprising:
electronically transmitting a natural language prompt regarding food safety or nutritional labeling from the user to a computer system that is configured to:
send a response request to a trained large language model based on the transmitted natural language prompt regarding food safety or nutritional labeling, wherein the trained large language model has been trained with a data training set including food safety or nutritional labeling reference data;
receive a natural language response from the trained large language model in reply to the response request; and
provide the natural language response to the user; and
electronically receiving the natural language response from the computer system.

12. The method of claim 11, wherein electronically transmitting the natural language prompt regarding food safety or nutritional labeling from the user to the computer system includes electronically transmitting the natural language prompt regarding food safety or nutritional labeling from the user to the computer system via a web application.

13. The method of claim 11, wherein the natural language prompt specifies a jurisdiction of inquiry.

14. The method of claim 13, wherein the specified jurisdiction of inquiry is a jurisdiction in which the user is located.

15. The method of claim 11, comprising providing to the computer system user feedback on the natural language response.

16. An apparatus comprising:
a processor-based computer system including a memory and a processor, the memory having computer-readable instructions that, when executed, cause the computer system to:
electronically receive a natural language prompt regarding food safety or nutritional labeling from the user via a network;
send a response request based on the received natural language prompt regarding food safety or nutritional labeling to a trained large language model, wherein the trained large language model has been trained with a data training set including food safety or nutritional labeling reference data;
receive a natural language response from the trained large language model in reply to the response request; and
provide the natural language response to the user via the network.

17. The apparatus of claim 16, wherein the memory has computer-readable instructions that, when executed, cause the computer system to shape the received natural language prompt regarding food safety or nutritional labeling.

18. The apparatus of claim 16, wherein the memory includes one or more memory devices of a cloud computing environment.

19. The apparatus of claim 16, wherein the memory includes a non-volatile memory device.
